(19) United States
(12) Patent Application Publication
(10) Pub. No.: US 2016/0041155 A1
(43) Pub. Date: Feb. 11, 2016

(54) BIOMARKER SENSOR ARRAY AND CIRCUIT AND METHODS OF USING AND FORMING SAME

(71) Applicant: Bharath TAKULAPALLI, Chandler,, AZ (US)

(72) Inventor: Bharath Takulapalli, Chandler, AZ (US)

(21) Appl. No.: 14/777,425

(22) PCT Filed: Mar. 17, 2014
(86) PCT No.: PCT/US14/30891

§ 371 Date: Sep. 15, 2015

Related U.S. Application Data
(60) Provisional application No. 61/787,881, filed on Mar. 15, 2013.

(51) Int. Cl. G01N 33/543; G01N 27/414

(52) U.S. Cl. CPC G01N 33/5438; G01N 27/4145

(57) ABSTRACT

The present disclosure relates to biomarker sensor arrays, to circuits including the sensor arrays, to systems including the arrays, and to methods of forming and using the arrays, circuits, and systems. The arrays, circuits, and systems can be used to detect a variety of materials, including chemical, biological, and radioactive materials. The arrays and circuits can be used for, for example, screening tests, disease diagnostics, prognostics and disease monitoring.

### CROSS-REFERENCE TO RELATED APPLICATIONS

This application claims the benefit of U.S. Provisional Patent Application Ser. No. 61/787,881, entitled FIELD EFFECT SENSOR ARRAYS AND METHODS OF FORMING AND USING THE SAME, and filed Mar. 15, 2013, the contents of which are hereby incorporated herein by reference to the extent such contents do not conflict with the present disclosure.

### TECHNICAL FIELD

The present disclosure generally relates to sensor arrays and circuits for detection of materials. More particularly, the disclosure relates to arrays of sensors suitable for detecting various materials, such as chemical, biological or radioactive materials, to circuits including one or more arrays and to methods of forming and using the arrays and circuits.

### BACKGROUND

Sensor systems that detect disease specific biomarkers such as proteins, nucleic acids, antibodies, peptides, PTMs, glycans, carbohydrates, metabolites, cells, and the like, are finding increased use in the field of disease diagnostics. The state of disease is generally thought of as a rational and often rigorous progression, over a period of time, of abnormality or perturbance triggered at the biomolecular or cellular level, initiated by endogenous or exogenous factors, which can culminate in a harmful, life threatening condition. Due to this, it may be possible to diagnose onset of disease in early stages of development (even before symptoms appear) by detecting disease specific biomarkers, enabling effective therapeutic interventions and cure. Owing to recent advances in genomics, proteomics, transcriptomics and metabolomics, early stage biomarkers have been identified for different cancers, diabetes, cardiac conditions, autoimmune diseases such as rheumatoid arthritis, Alzheimer's disease, and specific infectious diseases, such as H1N1, HPV, hepatitis B/C, HIV, West Nile, and the like. However, current state-of-art diagnostic products based on biomarker detection, such as PSA test and mammography screening, are not optimal. This is because such products tend to over-simplify the underlying basis for disease, inaccurately correlating presence/absence of few biomarkers to end-outcomes of a disease, resulting in high false positives and/or negatives, and over/under-diagnosis. Diseases, especially cancers, are complex and highly heterogeneous, with multiple subtypes and individual-specific pathologies, making early-diagnosis a technological challenge. To address the inherent biological complexity, more sophisticated system-biology approaches involving highly-multiplexed detection of biomarkers and other key biomolecules, which will provide a snapshot of the disease-state at the tissue level, organ level or whole body (patient) level and yield high-confidence early stage diagnostics are desired.

### SUMMARY OF THE INVENTION

Various embodiments of the present disclosure relate to biomarker sensor arrays and circuits. Exemplary sensor arrays disclosed herein can be applied in (1) disease screening, prediction: predicting susceptibility of an individual to various diseases based on biomarkers present in patients' blood, saliva, serum, plasma, other body fluids, cell/tissue extracts to detect pre-symptomatic disease signatures (2) disease diagnosis: detection of disease specific biomarkers, in confirmatory testing and monitoring (3) disease prognosis: based on diagnostic data collected over time, categorizing a patient's condition into disease sub-types, including patient-specific pathology and clinical presentation (4) personalized therapy: development of individual-specific intervention strategies based on inherent drug resistance in patients, physician's decisions on using single or a combination of available drugs and their optimal patient-specific dosage (5) disease monitoring: periodic monitoring of patient using post-therapy biomarker detection to ascertain and follow response to therapy, enabling timely response to adverse reactions and development of drug resistance. Sensor arrays disclosed herein can be used to detect biomolecules with high sensitivity and high specificity, which can be applied to multiplexed biomarker detection with low false positives and low false negatives. In addition, sensor arrays can be applied to high-throughput label-free drug discovery.

In accordance with exemplary embodiments of the disclosure, a sensor array includes one or more (e.g., a plurality of) sensor nodes, wherein each sensor node comprises one or more (e.g., a plurality of) sensor elements, and each sensor element comprises one or more sensor devices. Each sensor node can detect a biomarker. A first sensor element of a plurality of sensor elements can produce a first electrical response to the biomarker and a second sensor element of the plurality of sensor elements can produce a second electrical response to the biomarker. Using multiple sensor elements to detect the same biomarker and produce different electrical responses upon detection of the biomarker, allows for reliable detection of the biomarker. In accordance with various aspects of these embodiments, the sensor array is configured to detect multiple biomarkers. For example, each node of the sensor array can be configured to detect a biomarker. The sensor device can be, for example, a device selected from a group consisting of field effect sensors, electrochemical sensors, nanowire sensors, nanotube sensors, graphene sensors, magnetic sensors, giant magneto resistance sensors, nano ribbon sensors, polymer sensors, resistive sensors, capacitive sensors, and inductive sensors. In accordance with further examples of these embodiments, a first sensor node includes first sensor devices and a second sensor node includes the first sensor devices or second sensor devices, wherein the first sensor devices are a first device type and the second devices are a second device type. For example, the first device type can be an FET device and the second type can be an electrochemical sensor, or giant magneto resistance sensor (GMR). Exemplary FET devices include partially depleted sensors, accumulation mode sensors, fully depleted sensors, inversion mode sensors, sub-threshold sensors, p-channel sensors, n-channel sensors, intrinsic sensors, complementary CMOS sensors, enhancement mode sensors, and depletion mode sensors. The FET sensor devices may range from 1 nm to 100 nm in width, 100 nm to 1 micron in width, or from 1 micron to 100 microns in width, or from 100 microns to few millimeters in width. The length of FET sensor devices may range from 10 nm to 1 micron, or 1 micron to 500 micron, or 500 micron to few millimeters. The various sensor devices within a sensor node can include (e.g., be coated with) a unique chemical or biological or radiation sensitive layer, such as a monolayer, multi-layer, a thin film, a gel material, matrix material, a nanostructured material, a nano porous material, a meso porous material, a micro porous material, a nano patterned material, or a micro patterned material. For example, the sensor devices can be coated with material selected from the group consisting of proteins, antibodies, nucleic acids, DNA strands, RNA strands, peptides, organic molecules, biomolecules, lipids, glycans, synthetic molecules, post translation modified biopolymers, organic thin films, inorganic thin films, metal thin films, insulating thin films, topological insulator thin films, semiconductor thin films, dielectric thin films, scintillation material films, and organic semiconductor films. By way of examples, all of the one or more sensor devices can be field effect sensor devices or other type of sensor device, wherein a plurality of sensor devices in any sensor element have the same features, wherein sensor elements in any sensor node have distinct features, wherein features of distinction between sensor elements include, for example, one or more features selected from a group consisting of semiconductor channel thickness, semiconductor channel doping, semiconductor channel implantation type and density, semiconductor channel impurity type, semiconductor channel impurity doping density, semiconductor channel impurity energy level, semiconductor channel surface chemistry treatment, semiconductor channel bias condition, semiconductor channel operational voltages, semiconductor channel width, semiconductor channel top thin film coatings, and semiconducting channel annealing conditions.

In accordance with further exemplary embodiments of the disclosure, sensors devices are formed using CMOS semiconductor technology (e.g. microfabrication technology). The one or more sensor devices can be formed on a substrate that is selected from the group consisting of silicon, silicon on insulator, silicon on sapphire, silicon on silicon carbide, silicon on diamond, gallium nitride, gallium nitride on insulator, gallium arsenide, gallium arsenide on insulator, and germanium, and germanium on insulator.

In accordance with additional embodiments of the disclosure, a sensor array for detecting biological, chemical or radioactive species includes a substrate, an insulator formed overlying selected portions of the substrate, and a plurality of semiconducting channels formed overlying the insulator. Each semiconducting channel in the plurality of semiconducting channels can include distinct features from at least one other semiconducting channel. Features of distinction/difference between the semiconducting channels can be selected from one or more of the group consisting of, for example, semiconductor channel thickness, semiconductor channel doping, semiconductor channel implantation type and density, semiconductor channel impurity type, semiconductor channel impurity density, semiconductor channel impurity energy level, semiconductor channel surface chemistry treatment, semiconductor channel bias condition, semiconductor channel operational voltages, semiconductor channel width, semiconductor channel top thin film coatings, and semiconducting channel annealing conditions. The plurality of semiconductor channels can be coated with a thin film or a monolayer or a multilayer of material. The plurality of semiconductor channels in the nested array can be configured to detect a single or multiple chemical or biological or radioactive species. Further, the array can be configured to detect a single or multiple chemical or biological or radioactive species. The plurality of semiconductor channels can be coated with one or more of a chemical or biological or radiation sensitive layer. The layer of one or more of a chemical or biological or radiation sensitive layer can be, for example, a monolayer, multi-layer or a thin film, a gel material, matrix material, a nanostructured material, a nano porous material, a meso porous material, a micro porous material, a nano patterned material, or a micro patterned material. The substrate can be selected from the group consisting of silicon, silicon on insulator, silicon on sapphire, silicon on silicon carbide, silicon on diamond, gallium nitride, gallium nitride on insulator, gallium arsenide, gallium arsenide on insulator, germanium, and germanium on insulator. The semiconductor channels may be coated with a dielectric thin film layer such as oxide, which can be coated with a chemical or biological or radiation sensitive layer or multiple layers; the layer or multiple layers can be selected from group consisting of, but not limited to, proteins, antibodies, nucleic acids, DNA strands, RNA strands, peptides, organic molecules, biomolecules, lipids, glycans, synthetic molecules, post translation modified biopolymers, organic thin films, inorganic thin films, metal thin films, insulating thin films, topological insulator thin films, semiconductor thin films, dielectric thin films, scintillation material films, and organic semiconductor films.

In accordance with further embodiments of the disclosure, a sensor system comprises an array as disclosed herein. The sensor system can also include microfluidic channels. For example, the microfluidic channels can be formed addressing each sensor channel individually or addressing multiple sensor channels, wherein microfluidic channels allow transferring fluidic materials to some or all sensor channels in the array of nested sensor arrays. The system can also include one or more of: A/D converters, relays, switches, amplifiers, comparators, differential circuits, source units, sense circuits, logic circuits, microprocessors, memory, FPGAs, batteries, and analog and digital signal processing circuits.

In accordance with further exemplary embodiments of the disclosure, methods of using an array, such as an array as described herein, comprises using the array for one or more of disease screening and diagnosis, such as for detecting biomarkers in a test medium such as, but not limited to, blood, serum, urine, sputum, cell extract, tissue extract, cerebrospinal fluid, saliva, plasma, and biopsy sample. Exemplary methods can include one or more of pattern recognition algorithms and disease signature approach to improve selectivity and specificity and predictive value of test.

In accordance with yet further exemplary embodiments of the disclosure, a circuit include an array as described herein the circuit can additionally include one or more of: A/D converters, sense/logic circuits, amplifiers, signal processing devices, FPGAs, relays, switches, processors, and memory.

### BRIEF DESCRIPTION OF THE DRAWINGS

A more complete understanding of exemplary embodiments of the present disclosure can be derived by referring to the detailed description and claims when considered in connection with the following illustrative figures.

FIG. 1 illustrates an array in accordance with exemplary embodiments of the disclosure.

FIG. 2 illustrates an exemplary sensor device in accordance with embodiments of the disclosure.

FIG. 3 illustrates an FET sensor response to SRC kinase auto-phosphorylation in accordance with exemplary embodiments of the disclosure.

FIG. 4 illustrates an FET sensor response to pH: threshold voltage variation plotted against pH value of buffer solution for four different fully depleted FET sensor devices in accordance with exemplary embodiments of the disclosure.

FIG. 5 illustrates sensor devices in accordance with further exemplary embodiments of the disclosure.

FIG. 6 illustrates an exemplary sensor node in accordance with yet further exemplary embodiments of the disclosure.

FIG. 7 illustrates an array in accordance with further exemplary embodiments of the disclosure.

FIG. 8 illustrates a response from a single sensor node to detection of a single test analyte in accordance with exemplary embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve the understanding of illustrated embodiments of the present disclosure.

### DETAILED DESCRIPTION

The description of embodiments provided below is merely exemplary and is intended for purposes of illustration only; the following description is not intended to limit the scope of the disclosure or the claims. Moreover, recitation of multiple embodiments having stated features is not intended to exclude other embodiments having additional features or other embodiments incorporating different combinations of the stated features.

The following disclosure provides improved sensor arrays, circuits including one or more arrays, systems including one or more arrays, and methods of forming and using the sensor arrays, circuits, and systems.

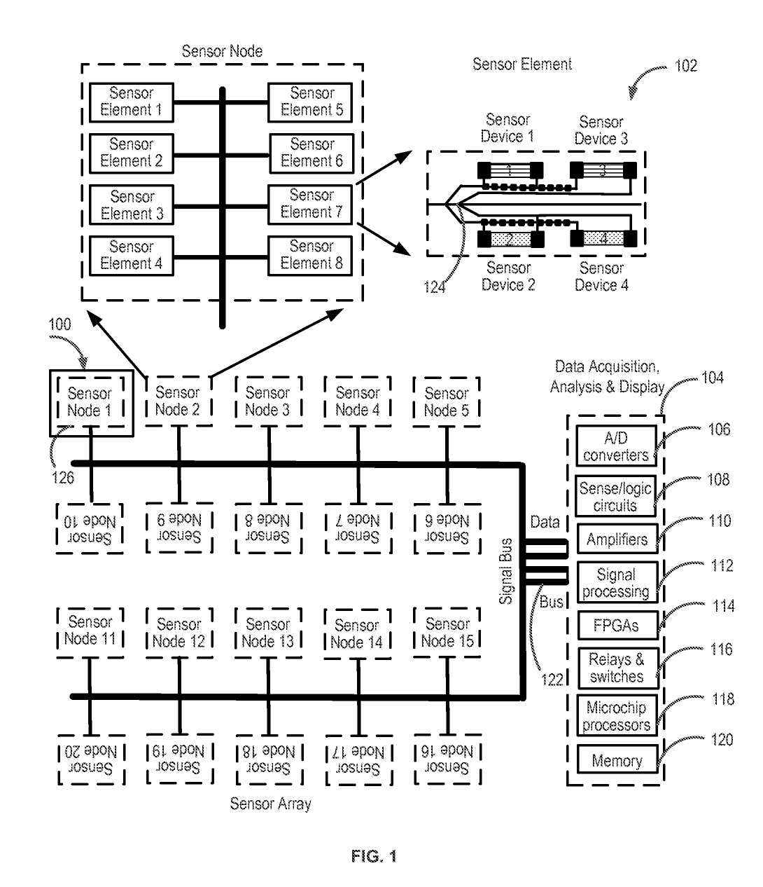

FIG. 1 illustrates a sensor array 100 in accordance with various embodiments of the disclosure. In the illustrated example, sensor array 100 includes a plurality of sensor nodes, illustrated as sensor nodes 1-20. Each sensor node includes a plurality of sensor elements. In the example, sensor node 2 (or all sensor nodes 1-20) includes sensor elements 1-8. Each sensor element includes one or more sensor devices, such as sensor devices 1-4. The sensor element can also include a reference electrode 124 for solution biasing.
1. The sensor device can be a single physical sensor device or sensor unit, in any array of sensors. Exemplary sensor devices can be, for example, device selected from a group consisting of field effect sensors, electrochemical sensors, nanowire sensors, nanotube sensors, graphene sensors, magnetic sensors, giant magneto resistance sensors, nano ribbon sensors, polymer sensors, resistive sensors, capacitive sensors, and inductive sensors. By way of example, one or more of the sensor devices can include a field effect transistor nanowire n-channel enhancement-mode fully depleted inversion-based device. By way of another example sensor device an include field effect transistor sensors such as the devices disclosed in application Ser. No. 12/663,666, entitled NANO STRUCTURED FIELD EFFECT SENSOR AND METHODS OF FORMING AND USING SAME, and filed Dec. 8, 2009, the contents of which are hereby incorporated herein by reference, to the extent such contents do not conflict with the present disclosure. By way of yet another example, sensor devices can include field effect transistor sensors, microwires and nanowire devise as discussed in report titled “Molecular sensing using monolayer floating gate, fully depleted SOI MOSFET acting as an exponential transducer” by Bharath Takulapalli, in journal ACS Nano Feb. 23 2010, 4(2): 999-1011, the contents of which are hereby incorporated herein by reference, to the extent such contents do not conflict with the present disclosure. Sensor devices may be a FDEC charged coupled sensor or potential coupled sensor or any other field effect sensor, micro scale devices or nanowire devices. Another example sensor device includes an electrochemical sensor with surface structure.

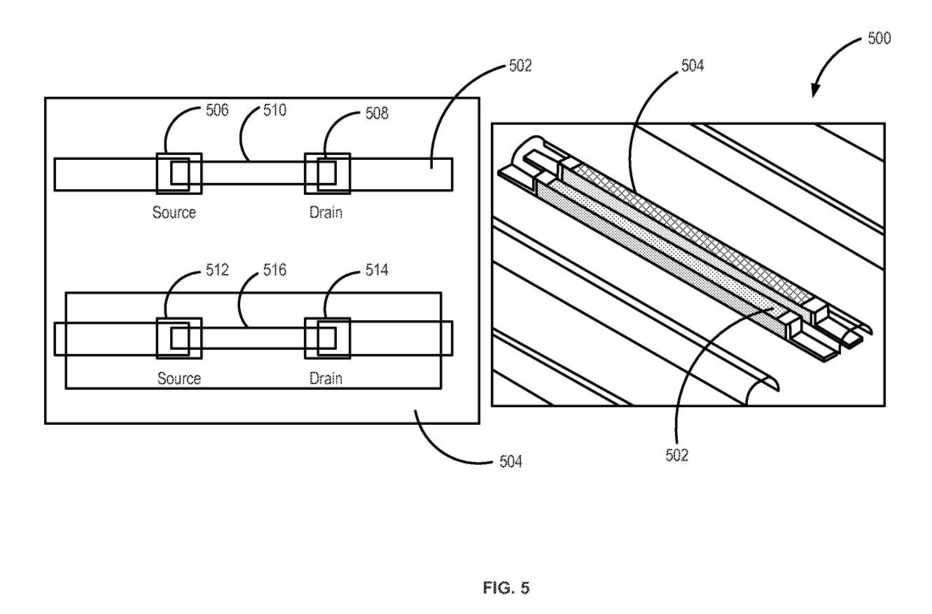

Each sensor element includes at least one sensor device. Examples: sensor element might comprise 1 sensor device, 2 sensor devices, 4 sensor devices, 8 sensor devices, or the like. In an exemplary embodiment a sensor element includes at least 2 sensor devices where one sensor device is an active device that functions to sense a target analyte and a second sensor device that is a reference device that does not aim to detect the analyte, but rather measures a background signal. In another example, a sensor element comprises at least 4 sensor devices, where two sensor devices are active devices such as n-channel and p-channel CMOS field effect transistor sensors and another two sensor devises are in-active versions of n-channel and p-channel sensors acting as reference devices. In one exemplary embodiment sensor element comprises at least two sensor devices connected in a differential or comparator circuit. Such exemplary sensor devices and circuits are discussed in more detail below in connection with FIG. 5.

As noted above, sensor elements can include reference electrode 124. Reference electrode 124 can be used in combination with sensor devices in the sensor element, for purposes of referencing solution bias in liquid phase experiments. An example sensor electrode can be a metal electrode, such as a platinum electrode.

Sensor nodes include at least one sensor element. Examples: sensor node might comprise 1 sensor element, 2 sensor elements, 4 sensor elements, 8 sensor elements, 16 sensor elements, 32 sensor elements, 100 sensor elements, or the like. Each of the sensor elements in a sensor node can have different features from at least one another sensor element in the node, and in some cases have different features from all other sensor elements in the node. Due to differing features, a first sensor element of the plurality of sensor elements can produce a first electrical response to the biomarker and a second sensor element of a plurality of sensor elements can produce a second electrical response to the biomarker. An exemplary sensor node includes sensor elements including one or more field effect transistor sensor devices (micro sensor or nano sensors), wherein sensor devices in sensor element-1 are operated in fully depleted regime with inversion, sensor devices in sensor element-2 are operated in partially depleted regime with inversion, sensor devices in sensor element-3 are operated in fully depleted regime in sub-threshold region, sensor devices in sensor element-4 are operated in partially depleted regime in sub-threshold region, sensor devices in sensor element-5 are operated in fully depleted regime in accumulation, sensor devices in sensor element-6 are operated in partially depleted regime in accumulation, sensor devices in sensor element-7 are operated in volume accumulation mode, sensor devices in sensor element-8 are operated in volume inversion mode, another set of eight sensor elements from 9-16 wherein the sensor devices in these sensor elements are operated in depletion-mode versus enhancement-mode operation in sensor elements 1-8. Various combinations of these sensor devices and various numbers of devices are within the scope of this disclosure.

Exemplary sensor nodes (or each sensor device within a sensor node) can be coated with a sensitive layer or a multi-layer (e.g., a unique sensitive layer) to detect a single target analyte or species. For example, a sensor node (or devices within the node) can be coated with a monolayer, multi-layer or thin film of biochemical materials (antibody-1) to detect a specific disease biomarker (antigen-1), where sensor node detects a unique bio-chemical interaction of disease biomarker (antibody-1 binding with antigen-1). In an example embodiment, a sensor node includes 16 sensor elements, which each comprise 4 sensor devices each, may be applied to detecting a single biochemical interaction (e.g., antibody-1 binding with antigen-1). In accordance with exemplary aspects of these embodiments, each sensor device in the sensor node is capable of detecting the same target analyte, but using different types of sensor devise. The different types of devices can use different modes of detection, whereby the cumulative of the detection signals, combinatorial sensor array response, results in high specificity detection of target analyte or disease biomarker. A second sensor node can be coated with a different sensitive biochemical material (antibody-2) and applied to detecting the same specific biomarker (antigen-1), where the second sensor node detects a second unique biochemical interaction of the disease biomarker (antibody-2 binding with antigen-1). Multiple sensor nodes can be applied to detecting a single disease biomarker. And, multiplicity of sensor nodes may be applied to detecting multiplicity of biomarkers. Exemplary sensor nodes can be used for high specificity detection of a single target analyte by combinatorial detection of target analyte interaction using sensor devices of different types that measure a single bio-chemical interaction (e.g., antigen-antibody interaction).

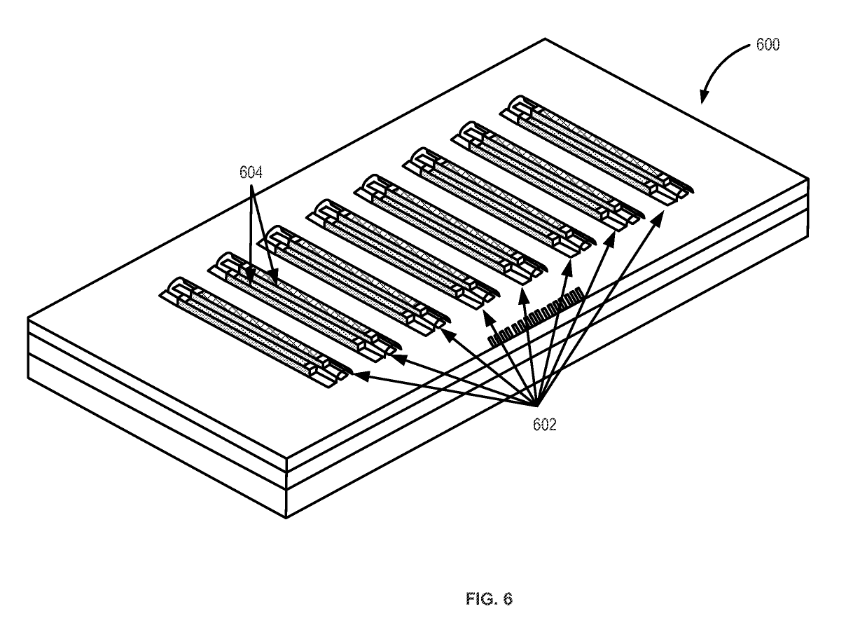

FIG. 6 illustrates an exemplary sensor node 600, which includes eight sensor elements 602, each comprising two sensor devices 604. In the illustrated example, each sensor element 602 has different device features compared to other sensor elements, which might result in different electric response when used to detect a given (same) chemical or biomolecular or radiological species. All sensor elements 604 in node 600 can be modified with a single chemical or biological or radiological sensitive thin film.

Sensor arrays, such as sensor array 100, include at least one sensor node. Arrays, such as array 100 are configured to detect at least one analyte in a test medium. A sensor array might comprise 10 sensor nodes, 20 sensor nodes, 100 sensor nodes, 1000 sensor nodes or 10,000 sensor nodes or 100,000 sensor nodes or 1 million sensor nodes of 10 million sensor nodes, 100 million sensor nodes, or any suitable number of sensor nodes.

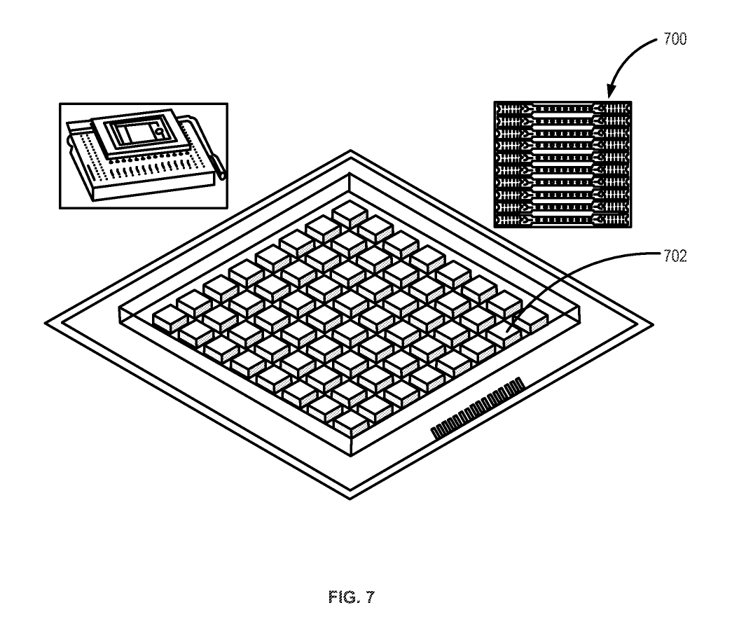

FIG. 7 illustrates another sensor array 700 in accordance with further exemplary embodiments of the disclosure. Sensor array 700 includes 10×10 sensor nodes 702. Each sensor node 702 can be configured to detect a different biomarker. In addition, more than one sensor node 702 can be configured to detect a single target biomarker. Each of sensor nodes 702 can be coated with chemical or biological sensitive films or materials that interact differently with the target biomarker. Each of the sensor nodes 702 may be packaged encapsulated as needed in wells, nano-cells, enclosed areas. Each of nodes 702 can be electrically addressable individually or all at a time, sequentially or randomly, to extract sensing signals.

Sensor signal acquisition in sensor array 100 or 700 can done using transistor switches. The size of sensor array may be 1 square millimeter or around 1 square centimeter or around 10 sq. centimeter square or 25 sq. centimeters square or 100 centimeter square or 200 centimeter square or 1000 centimeter square. In a given array of sensors, sensor devices or sensor elements or sensor nodes may be used once for a single sensing application or may be reused for multiple sensing events, wherein all or few sensor devices or sensor elements or sensor nodes may be used simultaneously, or may be used sequentially progressing to using the next in serial fashion only after using the previous one, or in parallel fashion in groups of sensor elements, or in any random fashion.

Sensor arrays in accordance with various examples of the disclosure can be configured as a Redundant Combinatorial Detection Array (RCDA). In an exemplary case an RCDA array performs as a sensor node in a nested sensor array comprising plurality of sensor nodes. Redundant Combinatorial Detection Array is a sensor array that can increase the selectivity of device response in detection of a specific target species. In an RCDA sensor array all the sensor devices are designed and fabricated with similar surface physical and chemical functionalities to detect the unique target species. The difference between each device element is mainly in the device functionality attributed by differences in doping density, device thickness, regime of operation (enhancement mode, depletion mode, partial depletion with inversion mode and accumulation mode etc.), carrier type (n-channel vs p-channel, electrons vs holes), different semiconducting channel layers, different semiconductor material and such. Example varying parameters include, but not limited to, semiconductor channel thickness, semiconductor channel material, semiconductor channel doping, semiconductor channel implantation type and density, semiconductor channel impurity type, semiconductor channel impurity doping density, semiconductor channel impurity energy level, semiconductor channel surface chemistry treatment, semiconductor channel bias condition, semiconductor channel operational voltages, semiconductor channel operations bias, semiconductor channel width, semiconductor channel top thin film coatings, semiconducting channel annealing conditions. The differences may also be in interface or bulk or impurity trap or defect state density, and their location in energy band gap. Exemplary different device elements with differing attributes can be designed and fabricated using semiconductor (e.g., ULSI) fabrication technology. Further examples of these embodiments obtain an output sensor signal that is ultra-highly selective, and hence gives relatively low amounts of false positives, and also decrease false negatives due to high sensitivity.

One simple example of RCDA array is a CMOS pair: enhancement mode n-channel and depletion mode p-channel devices, with same surface physical and chemical functionality. When the CMOS pair includes FDEC device elements, a negative charge addition on the device surface (due to target species binding) causes enhancement mode n-channel FDEC device element to increase in drain current, while the same negative charge addition causes a decrease in drain current in the second device—a depletion mode p-channel FDEC device. Similarly another CMOS pair: depletion mode n-channel and enhancement mode p-channel devices can be used for selective detection of positive charge addition on device surface due to target species interaction. This simple array of 4 CMOS FDEC device elements constitutes an example RCDA for selective detection of specific target species. Each of these 4 device elements can be configured in a differential pair circuit each, with respective reference/control devices, to make a RCDA of eight device elements. Or alternately a complementary pair of n-channel and p-channel devices that are biased in weak inversion or sub-threshold region can be used to detect negative charge and positive charge additions at the same time. Response of one device is expected to be opposite to the response of other device, for positive or negative charge additions.

In this RCDA example the devices mentioned are fully depleted FET sensor devices, which is not a necessary limitation. By controlling the thickness of the semiconductor channel layer and the doping density, it is possible to operate the devices in full volume inversion mode or in partial depletion mode of full depletion mode of the semiconductor thin film integrated into an RCDA for increasing selectivity of detection. The device can be operated in accumulation mode or in depletion mode or in inversion mode. Another example embodiment of RCDA array is tabulated below:

| | Response of device to target species |
|---|---|
| | interaction or change in positive (+ve)/ |
| Device Element description | negative (−ve) charge on device surface |
| Enhancement mode n-channel FDEC device | Addition of −ve charge causes exponential |
| | increase in drain current |
| Depletion mode p-channel FDEC device | Addition of −ve charge causes exponential |
| | decrease in drain current |
| Depletion mode n-channel FDEC device | Addition of +ve charge causes exponential |
| | decrease in drain current |
| Enhancement mode p-channel FDEC device | Addition of +ve charge causes exponential |
| | increase in drain current |
| Depletion (or enhancement or both) mode n- | Addition of −ve charge causes exponential |
| channel Ultra-thin volume inversion device | decrease in drain current and vice versa |
| (or partial depletion of film) | |
| Depletion mode (or enhancement or both) p- | Addition of +ve charge causes exponential |
| channel Ultra-thin volume inversion device | decrease in drain current and vice versa |
| (or partial depletion of film) | |
| Depletion mode (or enhancement or both) | Addition of +ve charge causes exponential |
| Ultra-thin p-type device operated in | decrease in drain current and vice versa |
| Accumulation | |
| Depletion mode (or enhancement or both) | Addition of −ve charge causes exponential |
| Ultra-thin n-type device operated in | decrease in drain current and vice versa |
| Accumulation | |

An example RCDA array such as above may contain 8 sensor elements in respective differential pairs—a total of 16 sensor devices, where the RCDA may comprise an example sensor node. Further different devices types of sensor elements can be added to the above to increase the selectivity of sensor array, e.g., to decrease false positives. Or multiple devices of one or more types listed above can be included to provide further redundancy in signal measurement. A single RCDA array can constitute up to a hundred or more sensor devices. Such high level of redundancy becomes useful when dealing with detection scenarios involving biomarker detection in detection of disease, cancer, etc. in vivo and in vitro diagnostics; in chemical and manufacturing industry for process control, in food industry etc., toxic gas or nuclear or radiation sensing in mass transport systems, malls, public gatherings etc. High level of redundancy is beneficial in scenarios where false positives are not desirable, or are prohibitively costly. At the same time such highly redundant RCDA sensor devices can be fabricated on a single chip in an inexpensive manner, providing maximum value in such scenarios.

For FET sensor devices, more particularly FDEC sensor devices, one of the most important aspect parameters is the trap states (interface or bulk or impurity or other kinds of trap states). The nature of defect states, density of interface traps states, location of these traps states within the semiconductor bandgap, and such others are important parameters for FDEC sensor device performance and operation. A Differential Combinatorial Detection Array (DCDA) is an array of FET sensor devices wherein each Sensor device of the array differs from at least one another sensor in either of the two ways (or both ways): (1) by way of using a different surface chemical or physical functionalization or different dielectric, semiconducting layers on active area of each ‘sensor element’ or (2) by way of using different interface trap parameters or bulk trap parameters or impurity trap parameters or other interface, bulk defect states or other semiconductor material parameters for each of the sensor elements within the array. Engineering trap state energy level: It is possible to approximately control the physical localization and energy location of trap states in the semiconductor band gap by controlling the nature of impurity doping in the bulk or at the interfaces. A sensor array consisting of sensor devices or sensor elements with each having different interface trap states that have peak densities at 0.1 eV, 0.2 eV, 0.3 eV, 0.4 eV, 0.5 eV, 0.6 eV, 0.7 eV, 0.8 eV, or 0.9 eV below the conduction band of the semiconductor channel material, forms a DCDA array. Each of the sensor elements with different trap state densities, energies, respond differently to interactions due to different target species.

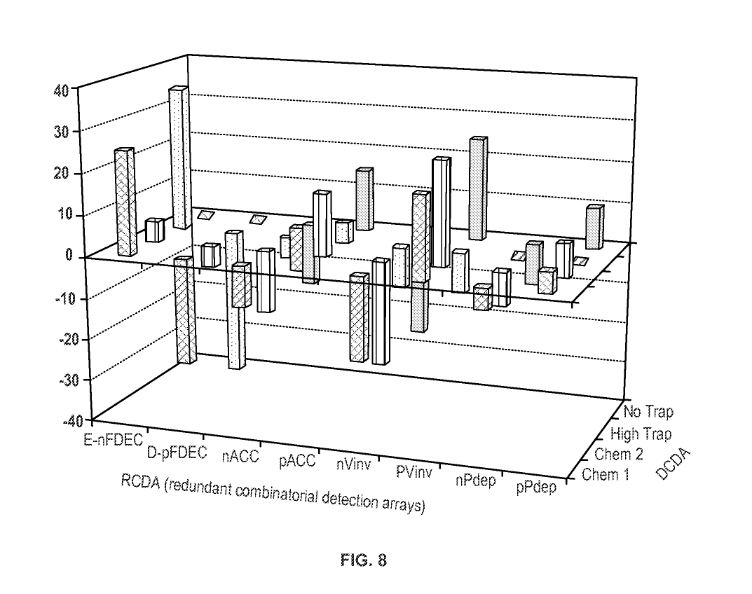

FIG. 8 illustrates a response from a single sensor node comprising an RCDA DCDA array to detection of a single test analyte. Test analyte might be a disease biomarker, a molecule, radiation, ions or other species of interest. Each sensor element in the sensor node has features differing from other sensor elements in the node, which might result in a different electric response from the sensor elements for a given (same) target analyte detection. Sometimes the responses from each sensor device in the node can be pre-determined, or expected to increase or decrease with certain amplitudes, for a given charge or potential or chemical or biological or radiological interaction with the sensitive device or device surface. In one example, all sensor elements and sensor devices in the node may be coated with a single chemical or biological or radiological sensitive material.

Sensor arrays as disclosed herein can be used as electronic nose and electronic tongue applications. Such arrays may contain from single or couple of sensor nodes up to millions of sensor nodes, where each sensor node may comprise 100 sensor elements, where each sensor element may comprise 32 sensor devices, forming a nested supra-array of sensor devices. These sensor elements might be a combination of DCDA and, or RCDA or any other similar sensor element architectures, nested one within the other, or in discrete fashion, depending on the application of the final field effect sensor arrays. All these sensor array applications include sensor devices that are in general any kind/type of field effect sensor or other kinds of sensors listed in the text here.

Sensor arrays in accordance with addition examples of the disclosure can include a reference-less sensor array configuration for pH sensor applications. Almost all biological processes, biochemical reactions in living cells and organisms occur in aqueous conditions, in the presence of water which acts as a solvent, catalyst, reactant etc. So the concentration of hydrogen ions ([H⁺] or [H₃O⁺] hydronium ions) inside human body is a physiological parameter of body functionality, from functioning of various organs to functioning of different organelle inside of cells. The importance of pH, calculated as the negative logarithm of hydrogen ion concentration, as a parameter at the intracellular level, inter-cellular or tissue level, at the organ level and for evaluating activity of body fluids, specifically blood is well established. In the sub-cellular case, local pH drastically affects vital cellular processes and any deviation of pH from the normal leads to loss of enzymatic functionality, up-regulation or down-regulation, inhibition, denaturing and digestion of cellular components, cell disease and eventually cell death. The human body maintains proper pH balance (pH 7.35 in blood) through acid-base homeostasis, to prevent build-up of acidic (or basic) species at any specific location inside the body. A decrease in pH of blood below 6.8 or an increase in pH above 7.8 may result in death. Due to the central role played by hydrogen ion concentration in many biological processes, spatial and temporal monitoring of pH in vivo at specific points inside human body is of significant clinical interest.

Inadequate supply of insulin in diabetics limits cell metabolism and increases glucose concentrations in blood, resulting in an increase in acidity. Build-up of ketone bodies through ketoacidosis occurs in Type I diabetes, indicated by lowering of blood pH. Variation of blood pH from the normal, limits oxygen carrying capability of red blood cells leading to oxygen starvation in tissues. Muscle pH can be used to triage trauma victims and to indicate poor peripheral blood flow in diabetic patients. In case of cancer cells increased proliferation leads to production of large amount of adenosine triphosphate (ATP) and other acidic compounds from increased glucose metabolism. To prevent intra-cellular acidification, the excess hydrogen ions are transported out of cells leading to inter-cellular acidification in cancer tissue. By monitoring inter-cellular (tissue) pH in vivo or in vitro, response of cancer cell growth to therapeutic agents can be measured in time.

Since pH variation is at least partially brought about by cellular metabolism, i.e., energy conversion and respiration processes, another organ of interest in this discussion is human brain. A brain consumes a large amount of energy, over 25% of total energy in a human, and also requires about 20% of blood supply. As brain activity is heterogeneous and neuronal activity is region specific, local activity of brain corresponds to local appetite for energy and blood resulting in increased region-specific metabolism rate and cerebral blood flow. Hence accurate spatial and temporal monitoring of pH variations across the brain is expected to yield information of region-specific brain activity, metabolism rates and local blood flow characteristics. A major physical impact to the head can lead to brain injury, ischemia both of which result in a decrease of pH from the normal by 0.5 to 1 unit. Patients of traumatic injury or stroke are implanted with sensors introduced percutaneously, allowing for continuous pH monitoring which assists in measuring effectiveness of therapy.

pH sensing for diagnosis of GERD: Chronic acid-reflux condition resulting in heartburn, regurgitation, irritation is diagnosed as gastroesophageal reflux disease (GERD, also GORD), and can cause tissue damage, esophagitis, etc. Another condition brought about by acidic pH in esophagus is Barrett's esophagus which is believed to be major risk factor in development of esophageal adenocarcinoma that ranks sixth in cancer mortality. GERD is caused by abnormal functioning of lower esophageal sphincter (LES), where acid reflux (and non-acid reflux) occurs from stomach back into the esophagus, resulting in pH change over a wide range, from pH7 (normal) to pH2 (very acidic). Reflux condition is diagnosed as GERD when pH falls from pH 7 to below pH 4 abruptly (within 30 seconds) and remains below pH 4 for a significant period of time, as characterized by Johnson and DeMeester (JD) score well above normal (14.72). In addition to manometry for pressure testing of LES, pH sensing has been accepted as the gold standard for GERD diagnosis. Other than pH, multiple intraluminal electric impedance (MII) based measurements have also been used for GERD diagnosis, often in combination with integrated pH monitoring (MII-pH). Monitoring of esophageal pH is traditionally done at a-point 5 cm above LES, while monitoring at other distal locations such as 15 cm above LES and 10 cm below LES into stomach are also used in combination. While there are many catheter-based and capsule pH sensor technologies available using polymer films, fluorescent detection, optical fibers, ISFETs, near infra-red (NIR) and NMR, the most accepted standard in GERD diagnosis is ambulatory pH testing using wireless capsule sensors (tubeless). One wireless pH sensor capsules is Medtronic's Bravo pH monitoring system that simultaneously measures pH and transmits data using radio telemetry, from 24 hours up-to 4 days. A FDEC FET Nanowire pH sensor device in an sensor element and sensor array as disclosed herein can address these problems in clinical application of pH sensors for GERD diagnosis, and can be used either in capsule configuration or can be integrated on-chip with impedance sensors for combined MII-pH multiple intraluminal test configuration.

An array of FDEC FET sensor devices or other field effect sensor devices can be used for accurate measurement of pH of a solution at the point-of-use. This pH sensor may be operated with or without need for any kind of reference device working in parallel. The use of reference electrode or reference device in conventional pH sensor devices prohibits its wide use for a variety of applications, including in vitro and in vivo applications. FDEC device arrays coated with select top dielectric materials, chemical sensitive films, with varying surface chemical terminations and respective oxidation-reduction potentials can be used for sensing unique pH values of solutions. Due to the fact that FDEC charge coupling occurs at specific pH value of solution for a given surface chemistry of the device, these sensor arrays can be used as reference-less pH sensor devices. Native oxide has surface reactive hydroxyl groups that undergo ion exchange reactions between pH 6.5 and pH 7.5 (as an example pH point location).

FDEC sensor devices, when biased at predetermined potentials, exhibit varied response depending on the device structure, architecture, functioning and the pH value of the solution. Nested arrays that for example contain DCDA arrays of nested 16 element RCDA arrays (as example), with the differential parameter between the RCDA arrays being the surface functionalization, or different trap state characteristics. By using this as sensor node in this example, coating the surface of each RCDA array with unique, predetermined surface coating, of chemical or organic or inorganic thin film or of unique surface terminations, each of the RCDAs can be used to determine and distinguish, with or without external reference devices, between various pH values of solutions they are exposed to. A 14 DCDA array with nested RCDAs with 14 respective, chosen, selected, predetermined surface terminations, surface thin film coatings, can be used to distinguish between pH values from pH 1 to pH 14. These pH sensor arrays can be used multiple times, by pre and post treatments as cost effective devices. Also they can be used in-vivo for device implant applications, for measuring pH inside the body at various locations, or in general configured to measure other in-vivo biomarkers, inside of different organs.

Sensor arrays as disclosed herein can also be used to detect radioactive material. When light (electromagnetic radiation) with energy less than band-gap energy of a semiconductor is incident on the surface of semiconductor, photons interact with various trap states, forming donor-acceptor pair with respective states in conduction and valence bands—leading to photon absorption, and trapping of electrons/holes and hence forming of excess charges inside the semiconductor or at its interface. Characterization of photonic interactions of interface trap states in conventional FET sensor structures has been reported, but no studies in terms of detection of photons due to these interactions. When coupled with ‘fully depleted FET sensor structures’ the charges formed due to trap aided absorption produce an exponential device current response due to second order coupling with threshold voltage of the inversion channel, potentially acting as ‘ultra-low power photon/radiation detector.’ The same concept of trap coupling can be used to detect higher energy radiation by integration of scintillator material via detection of secondary emissions (Bremsstrahlung). The interaction of short range low energy radiation through electronic signatures obtained from barrier thin film coated integrated FET sensor devices, transistors can be applied to detect sub atomic particles (alpha, beta, low energy neutrons, others).

Interaction of high energy nuclear radiation, such as gamma rays, neutrons and other charged particles, with special scintillator materials produces photons of narrow band-width in the visible and near-UV regions of the electromagnetic spectrum. Photons emitted from these scintillator materials can be absorbed by the integrated fully depleted field effect devices, via trap-coupled photoexcitation to produce free charges in the fully depleted semiconductor region, which in-turn can be accurately detected by inversion channel modulation in field effect exponentially coupled transducers (capacitor or transistor). A small change in threshold voltage produces orders of magnitude variation in inversion current in depletion-biased devices. Hence inversion current response can be used to detect trap-assisted charge generation caused by nuclear radiation interaction. In nuclear radiation detection applications, threshold voltage variation is expected to be due to exponential charge coupling and also due to free carrier generation (work function coupling). Both trap-coupled charge generation (charge transduction) and free carrier generation (flush of carriers) are expected to contribute to exponential inversion current response, with the latter being a transient response. Nuclear radiation (gamma, neutron and other charge particle) interaction with semiconductor materials (HPGe) or certain scintillator materials (2 micron thick boron film coated on top of the device), produces electron-hole pairs as end-result of radiation energy loss to the material lattice. The produced electrons/holes can be captured on acceptor/donor impurity traps inside the fully depleted semiconductor. This trap assisted charge capturing generates new charge in the film along with complementary free charge carriers, both of which cause exponentially coupled field effect response in inversion channel conductance, as explained above.

Trap assisted Absorption of Photons: Absorption of photons via interface, bulk and impurity traps followed by detection can be done by silicon, AlGas, GaN, other III-V material, or compound semiconductor material based sensor devices, in field effect sensor devices in general and in FDEC sensor in particular. Nanostructured semiconductor surfaces, such as nanopores, nano gratings and nano pillars are expected to increase interaction cross section of incident radiation, other than aid beam (particle) collimation, resulting in increased trap assisted absorption characteristics. Barrier aided absorption of short range radiation via integration of above FET sensor devices with different barrier films with various surface nanostructures and thicknesses (metals, semiconductors and insulators, and combination of sandwich structures) can be applied to specific and combinatorial electronic signatures from trap aided absorption of dispersed energy and secondary radiation due to interaction with weak nuclear radiation. By integrating with scintillator materials electronic signatures to high energy radiation such as gamma/X rays, neutrons and such can be detected using field effect sensor devices. Novel nano and micro structures towards collimated optimized detection of secondary radiation, particle emissions will increase sensor sensitivity.

Referring again to FIG. 1, a sensor system 102 can include a sensor array, such as array 100. Sensor system 102 can also include additional circuit features to sense, relay, store, process and display information from sensor devices in the array, including information analysis, data correlation, calculation of recommendations and decisions. In an example embodiment, sensor devices in sensor system are addressed using VLSI transistor switch controlled parallel crisscross address lines architecture, similar to memory devices and computer microprocessors. The addressing architecture may comprise of stacks, segments, paging units, registers, kernels, blocks, which may be addressed in a nested addressing format. A sensor system may comprise circuit elements selected from one or more of, but not limited to, A/D converters, relays, switches, amplifiers, comparators, differential circuits, source units, sense circuits, logic circuits, microprocessors, memory, FPGAs, analog and digital signal processing circuits, and the like. In the example illustrated in FIG. 1, circuit elements 104 include A/D converters 106, sense logic circuits 108, amplifiers 110, signal processing devices 112, FPGAs 114, relays and switches 116, microchip processors 118, memory 120 and data bus 122.

Sensor system 102 or array 100 can include a sensor well 126 formed around one or more sensor nodes, as an isolated micro or nano well used to transfer, isolate and contain fluid substances, or to screen sensor devices from environment or noise or impurities which impede sensor function.

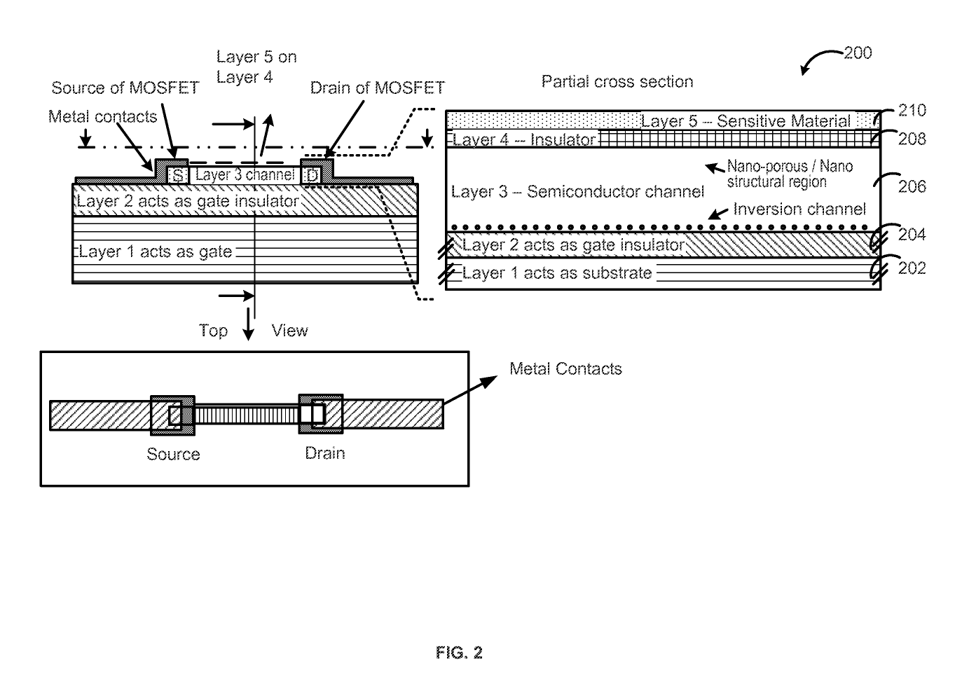

Turning now to FIG. 2, a device 200, suitable for a sensor device (e.g., sensor device 1-4 of array 100), is illustrated. Sensor device 200 includes a base 202, which can be or act as substrate, an insulator layer 204, which acts as a gate insulator, a channel region 206, which acts as a semiconductor channel, and a dielectric layer 208, which acts as an insulator. Device 200 can also include a sensitive material layer 210.

Base 202 acts as a gate during sensor 200 operation. Base 202 may be formed of any suitable material. Examples include, but are not limited to metals and metal nitrides such as Ge, Mg, Al, Sc, Ti, V, Cr, Mn, Fe, Co, Ni, Cu, Zn, Ga, Y, Zr, Nb, Mo, Ru, Rh, Pd, Ag, La, Hf, Ta, W, Re, Os, Ir, Pt, Au, TaTi, Ru, HfN, TiN, and the like, metal alloys, semiconductors, such as Group IV (e.g., silicon) Group III-IV (e.g., gallium arsenide) and Group II-VI (e.g., cadmium selenide), metal-semiconductor alloys, semi metals, or any organic or inorganic material that acts as a MOSFET gate.

A thickness of base 202 may vary according to material and application. In accordance with one example, base 202 is substrate silicon in silicon-on-insulator (SOI) wafer. In another example, base 202 is a flexible substrate, for example, an organic material, such as Pentacene.

Insulator layer 204 acts as a gate insulator or gate dielectric during operation of sensor 200. Layer 204 may be formed of any suitable material, such as any suitable organic or inorganic insulating material. Examples include, but are not limited to, silicon dioxide, silicon nitride, hafnium oxide, alumina, magnesium oxide, zirconium oxide, zirconium silicate, calcium oxide, tantalum oxide, lanthanum oxide, titanium oxide, yttrium oxide, titanium nitride, and the like. One exemplary material suitable for layer 204 is a buried oxide layer in an SOI wafer. A thickness of layer 204 may vary according to material and application. By way of one particular example, layer 204 is silicon oxide having a thickness from about 1 nm to 200 microns; in accordance with other aspects, layer 204 may be 1 mm or more.

Channel region 206 may be formed of a variety of materials, such as crystalline or amorphous inorganic semiconductor material, such as those used in typical MOS technologies. Examples include, but are not limited to, elemental semiconductors, such as silicon, germanium, diamond, tin; compound semiconductors, such as silicon carbide, silicon germanium, diamond, graphite; binary materials, such as aluminum antimonide (AlSb), aluminum arsenide (AlAs), aluminum nitride (AlN), aluminum phosphide (AlP), boron nitride (BN), boron phosphide (BP), boron arsenide (BAs), gallium antimonide (GaSb), gallium arsenide (GaAs), gallium nitride (GaN), gallium phosphide (GaP), indium antimonide (InSb), indium arsenide (InAs), indium nitride (InN), indium phosphide (InP), cadmium selenide (CdSe), cadmium sulfide (CdS), cadmium telluride (CdTe), zinc oxide (ZnO), zinc selenide (ZnSe), zinc sulfide (ZnS), zinc telluride (ZnTe), cuprous chloride (CuCl), lead selenide (PbSe), lead sulfide (PbS), lead telluride (PbTe), tin sulfide (SnS), tin telluride (SnTe), bismuth telluride (Bi₂Te₃), cadmium phosphide (Cd₃P₂), cadmium arsenide (Cd₃As₂), cadmium antimonide (Cd₃Sb₂), zinc phosphide (Zn₃P2), zinc arsenide (Zn₃As₂), zinc antimonide (Zn₃Sb₂), other binary materials such as lead(II) iodide (PbI₂), molybdenum disulfide (MoS₂), gallium selenide (GaSe), tin sulfide (SnS), bismuth sulfide (Bi₂S₃), platinum silicide (PtSi), bismuth(III) iodide (BiI₃), mercury(II) iodide (HgI₂), thallium(I) bromide (TlBr), semiconducting oxides like zinc oxide, titanium dioxide (TiO₂), copper(I) oxide (Cu₂O), copper(II) oxide (CuO), uranium dioxide (UO₂), uranium trioxide (UO₃), 6.1 Å materials or ternary materials, such as aluminium gallium arsenide (AlGaAs, AlxGa1-xAs), indium gallium arsenide (InGaAs, InxGa1-xAs), aluminium indium arsenide (AlInAs), aluminium indium antimonide (AlInSb), gallium arsenide nitride (GaAsN), gallium arsenide phosphide (GaAsP), aluminium gallium nitride (AlGaN), aluminium gallium phosphide (AlGaP), indium gallium nitride (InGaN), indium arsenide antimonide (InAsSb), indium gallium antimonide (InGaSb), cadmium zinc telluride (CdZnTe, CZT), mercury cadmium telluride (HgCdTe), mercury zinc telluride (HgZnTe), mercury zinc selenide (HgZnSe), lead tin telluride (PbSnTe), thallium tin telluride (Tl₂SnTe₅), thallium germanium telluride (Tl₂GeTe₅) and quaternary materials, such as aluminum gallium indium phosphide (AlGaInP, InAlGaP, InGaAlP, AlInGaP), aluminum gallium arsenide phosphide (AlGaAsP), indium gallium arsenide phosphide (InGaAsP), aluminium indium arsenide phosphide (AlInAsP), aluminum gallium arsenide nitride (AlGaAsN), indium gallium arsenide nitride (InGaAsN), indium aluminum arsenide nitride (InAlAsN), copper indium gallium selenide (CIGS), or quinary materials like gallium indium nitride arsenide antimonide (GaInNAsSb), and the like.

Channel Region 206 can also be made of organic semiconducting materials. Examples of such materials include, but are not limited to, polyacetylene, polypyrrole, polyaniline, Rubrene, phthalocyanine, poly(3-hexylthiophene, poly(3-alkylthiophene), α-ω-hexathiophene, Pentacene, α-ω-di-hexyl-hexathiophene, α-ω-dihexyl-hexathiophene, poly(3-hexylthiophene), bis(dithienothiophene, α-ω-dihexyl-quaterthiophene, dihexyl-anthradithiophene, n-decapentafluoroheptylmethylnaphthalene-1,4,5,8-tetracarboxylic diimide, α-ω-dihexylquinquethiophene, N,N′-dioctyl-3,4,9,10-perylene tetracarbozylic, CuPc, methanofullerene, [6,6]-phenyl-C61-butyric acid methyl ester (PCBM), C60, 3′,4′-dibutyl-5-5bis(dicyanomethylene)-5,5′-dihydro-2,2′:5′,2″terthiophene (DCMT), PTCDI-05, P3HT, Poly(3,3″-dialkyl-terthiophene), C60-fused N-methylpyrrolidine-meta-C12 phenyl (C60MC12), Thieno[2,3-b]thiophene, PVT, QM3T, DFH-nT, DFHCO-4TCO, BBB, FTTTTF, PPy, DPI-CN, NTCDI, F8T2—poly[9,9′ dioctylfluorene-co-bithiophene], MDMO-PPV—poly[2-methoxy-5-(3,7-dimethyloctyloxy)]-1,4-phenylenevinylene, P3HT—regioregular poly[3-hexylthiophene]; PTAA, polytriarylamine, PVT—poly-[2,5-thienylene vinylene], DH-ST—α,ω-Dihexylquinquethiophene, DH-6T—α,ω-dihexylsexithiophene, phthalocyanine, α-6T—α-sexithiophene, NDI, naphthalenediimide, F16CuPc—perfluorocopperphthalocyanine, perylene, PTCDA-3,4,9,10-perylene-tetracarboxylic dianhydrid and its derivatives, PDI—N,N′-dimethyl 3,4,9,10-perylene tetracarboxylicdiimide, or the like.

As noted above, in accordance with various embodiments of the invention, channel region 206 includes pores and/or structures to increase the device sensitivity.

Exemplary materials suitable for dielectric layer 208 include inorganic dielectric material that acts as a gate dielectric material. Examples include, but are not limited to, SiO₂, Si₃N₄, SiNx, Al2O₃, AlOx La2O₃, Y₂O₃, ZrO₂, Ta2O₅, HfO₂, HfSiO₄, HfOx, TiO₂, TiOx, a-LaAlO₃, SrTiO₃, Ta₂O₅, ZrSiO₄, BaO, CaO, MgO, SrO, BaTiO₃, Sc₂O₃, Pr₂O₃, Gd₂O₃, Lu₂O₃, TiN, CeO₂, BZT, BST, or a stacked or a mixed composition of these and/or such other gate dielectric material(s).

Dielectric layer 208 can additionally or alternatively include an organic gate dielectric material. Examples of organic materials include, but are not limited to, PVP—poly(4-vinyl phenol), PS—polystyrene, PMMA—polymethyl-methacrylate, PVA—polyvinyl alcohol, PVC—polyvinylchloride, PVDF—polyvinylidenfluoride, PαMS—poly[α-methylstyrene], CYEPL—cyano-ethylpullulan, BCB—divinyltetramethyldisiloxane-bis(benzocyclobutene), CPVP-Cn, CPS-Cn, PVP-CL, PVP-CP, polynorb, GR, nano TiO₂, OTS, Pho-OTS, various self assembled monolayers or multilayers or a stacked or a mixed composition of these and such other organic gate dielectric material.

Sensor device 200 can operate in depletion, accumulation or inversion, or transitioning from one to other, which includes all field effect transistor-based sensor devices and FDEC sensor devices, which may be a micro scale device or nano scale device or a nanostructured device or a combination of these. The semiconductor material might be organic semiconductor or inorganic semiconductor or a hybrid of both materials or in general any semiconducting material including graphene, carbon nanotubes, nanotubes of other materials, fullerenes, graphite, etc.

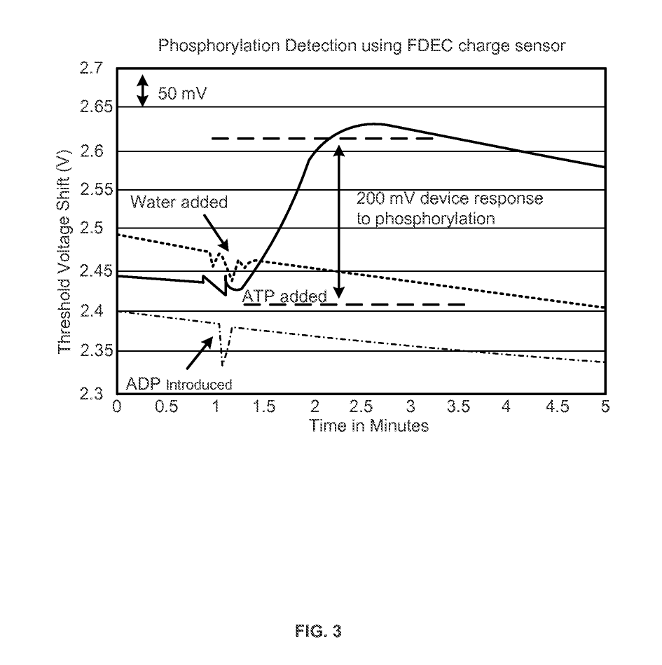

FIG. 3 illustrates an exemplary FET sensor device (e.g., sensor device 200) response to SRC kinase auto-phosphorylation. In the illustrated example, a large threshold voltage shift is produced in response to few pico moles of SRC protein immobilized on microbeads, upon addition 10 μl ATP. Addition of 10 μl aquilots of pure water and pure ADP produced no response.

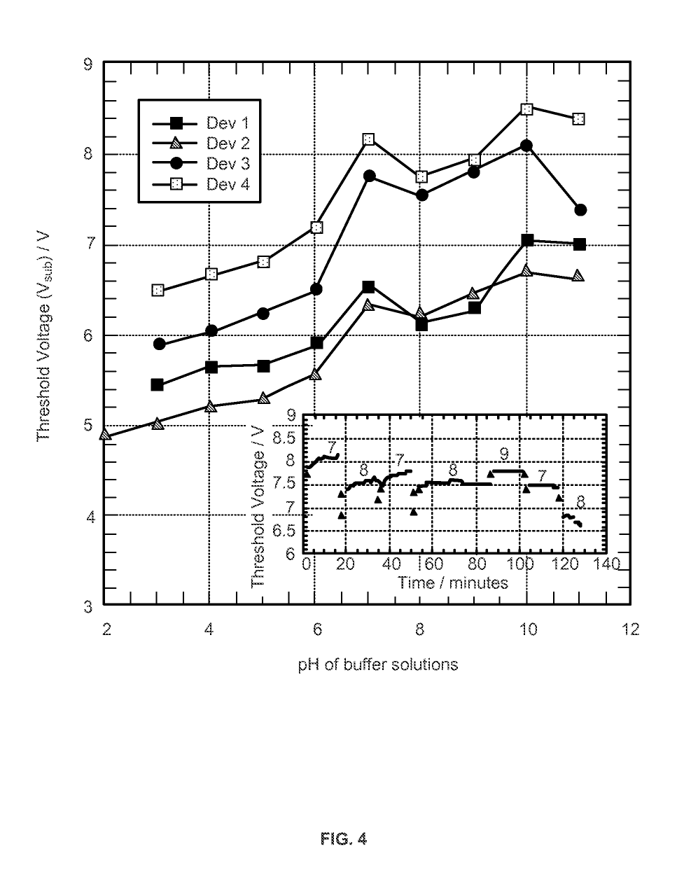

FIG. 4 illustrates a sensor device (e.g., sensor device 200) response to pH: Threshold voltage variation plotted against pH value of buffer solution for four different fully depleted FET sensor devices. All devices exhibit anomalous responses when transitioning from pH 8 pH 7 and from pH 11 to pH 10. In the inset is plotted device threshold voltage response vs. time, when the device is exposed alternately to pH 7 and pH 8 (also pH 9) buffer solutions. The anomalous response is seen both ways, from acidic to basic solutions and in the reverse order

Turning now to FIG. 5, a comparator (or differential pair) circuit 500 is illustrated. Circuit 500 includes a first sensor element 502 and a second sensor element 504. During operation of circuit 500, first sensor element 502 is exposed to target species, while second sensor element 504 is a reference device and is not exposed to target species. First and second sensor element can be connected in a differential circuit or similar other comparative circuit, which enables higher selectivity of target molecule detection, higher sensitivity by reducing the background noise. Circuit 500 enables higher selectivity of target species detection, higher sensitivity and higher selectivity by reducing the background noise, which may also be connected with an integrated amplifier circuit to increase the signal read out, or similar other electronic circuitry. In the illustrated example, sensor element 502 includes a source 506, a drain 508, and a channel region 510. Similarly sensor element 504 includes a source 512, a drain 514, and a channel region 516.

Target species (also referred to as target analyte) refers to any of the chemical or biological or explosive or nuclear or radiological species, or in general any mater, material or radiation the presence or absence of which in a medium is detected by using a sensor. This includes nano particles, single cells, multi-cells, organisms, virus, bacteria, DNA or proteins or macromolecules and cancer, disease biomarkers. The term target species also includes, for relevant sensing application, electromagnetic waves such as: visible light, infrared light, micro waves, radio waves, ultra violet rays, x rays, gamma rays, high energy electromagnetic radiation, low every electromagnetic radiation.

It is understood that the disclosed invention is not limited to the particular methodology, protocols and materials described as these can vary. It is also understood that the terminology used herein is for the purposes of describing particular embodiments only and is not intended to limit the scope of the present invention that will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A sensor array comprising:
a plurality of sensor nodes,
wherein each sensor node of the plurality of sensor nodes comprises a plurality of sensor elements, and each sensor element comprises one or more sensor devices,
wherein each sensor node detects a biomarker, and
wherein a first sensor element of the plurality of sensor elements produces a first electrical response to the biomarker and a second sensor element of the plurality of sensor elements produces a second electrical response to the biomarker.

2. The sensor array of claim 1, wherein the sensor array is configured to detect a plurality of biomarkers, wherein one or more of the sensor nodes of the plurality of sensor nodes detect one or more biomarkers.

3. The sensor array of claim 1, wherein the one or more sensor devices comprise a sensor device selected from a group consisting of field effect sensors, electrochemical sensors, nanowire sensors, nanotube sensors, graphene sensors, magnetic sensors, giant magneto resistance sensors, nano ribbon sensors, polymer sensors, resistive sensors, capacitive sensors, and inductive sensors.

4. The sensor array of claim 1, wherein a first sensor node comprises first sensor devices and a second sensor node comprises second sensor devices, wherein the first sensor devices are a first device type and the second sensor devices are a second device type.

5. The sensor array of claim 1, wherein the one or more sensor devices comprise field effect sensors.

6. The sensor array of claim 1, wherein the one or more sensor devices comprise electrochemical sensors.

7. The sensor array of claim 1, wherein the one or more sensor devices comprise giant magneto resistance (GMR) sensors.

8. The sensor array of claim 1, wherein each sensor node comprises a chemical or biological or radiation sensitive layer.

9. The sensor array of claim 1, wherein each sensor node comprises chemical or biological or radiation sensitive layer or multiple layers comprising material selected from the group consisting of proteins, antibodies, nucleic acids, DNA strands, RNA strands, peptides, organic molecules, biomolecules, lipids, glycans, synthetic molecules, post translation modified biopolymers, organic thin films, inorganic thin films, metal thin films, insulating thin films, topological insulator thin films, semiconductor thin films, dielectric thin films, scintillation material films, and organic semiconductor films.

10. The sensor array of claim 1, wherein the one or more sensor devices are produced using CMOS semiconductor technology.

11. The sensor array of claim 1, wherein the sensor devices are fabricated on a substrate that is selected from the group consisting of silicon, silicon on insulator, silicon on sapphire, silicon on silicon carbide, silicon on diamond, gallium nitride, gallium nitride on insulator, gallium arsenide, gallium arsenide on insulator, germanium, and germanium on insulator.

12. The sensor array of claim 1, wherein the one or more sensor devices in each sensor node are selected from the group consisting of partially depleted sensors, accumulation mode sensors, fully depleted sensors, inversion mode sensors, volume inversion mode sensors, volume accumulation mode sensors, sub-threshold sensors, p-channel sensors, n-channel sensors, intrinsic sensors, complementary CMOS sensors, enhancement mode sensors, and depletion mode sensors.

13. The sensor array of claim 1, wherein all of the one or more sensor devices are field effect sensors, wherein plurality of sensor devices in any sensor element have same features, wherein sensor elements in any sensor node have distinct features, wherein features of distinction between sensor elements is selected from a group consisting of semiconductor channel material, semiconductor channel thickness, semiconductor channel doping, semiconductor channel implantation type and density, semiconductor channel impurity type, semiconductor channel impurity doping density, semiconductor channel impurity energy level, semiconductor channel surface chemistry treatment, semiconductor channel bias condition, semiconductor channel operational voltages, semiconductor channel width, semiconductor channel top thin film coatings, and semiconducting channel annealing conditions.

14. A method of using the array of claim 1 for one or more of disease screening or diagnosis or prognosis or post-therapeutic monitoring.

15. The method of claim 14, wherein one or more of pattern recognition algorithms and disease signature approach are employed to improve selectivity.

16. A sensor array for detecting biological, chemical or radioactive species comprising:
a substrate;
an insulator formed overlying selected portions of the substrate; and
a plurality of semiconducting channels formed overlying the insulator,
wherein the each semiconducting channel in the plurality of semiconducting channels comprises features distinct from at least one another semiconducting channel, and
wherein the features are selected from the group consisting of semiconductor channel material, semiconductor channel thickness, semiconductor channel width, semiconductor channel length, semiconductor channel doping, semiconductor channel implantation type and density, semiconductor channel impurity type, semiconductor channel impurity density, semiconductor channel impurity energy level, semiconductor channel surface chemistry treatment, semiconductor channel bias condition, semiconductor channel operational voltages, semiconductor channel width, semiconductor channel top thin film coatings, and semiconducting channel annealing conditions.

17. The sensor array of claim 16, wherein the plurality of semiconductor channels are coated with one or more of a chemical or biological or radiation sensitive layer.

18. The sensor array of claim 16, wherein the substrate is selected from the group consisting of silicon, silicon on insulator, silicon on sapphire, silicon on silicon carbide, silicon on diamond, gallium nitride, gallium nitride on insulator, gallium arsenide, gallium arsenide on insulator, germanium, and germanium on insulator.

19. The sensor array of claim 16, wherein the semiconductor channels are coated with a chemical or biological or radiation sensitive layer, wherein the layer is a material selected from the group comprising of, but not limited to, proteins, antibodies, nucleic acids, DNA strands, RNA strands, peptides, organic molecules, biomolecules, lipids, glycans, synthetic molecules, post translation modified biopolymers, organic thin films, inorganic thin films, metal thin films, insulating thin films, topological insulator thin films, semiconductor thin films, dielectric thin films, scintillation material films, and organic semiconductor films.

20. The sensor array of claim 16, further comprising microfluidic channels, wherein the microfluidic channels are formed addressing each sensor channel individually or addressing multiple sensor channels, wherein microfluidic channels allow transferring fluidic materials to some or all sensor channels in the array of nested sensor arrays.

21-25. (canceled)
